(19) United States
(12) Patent Application Publication
(10) Pub. No.: US 2004/0247573 A1
(43) Pub. Date: Dec. 9, 2004

(54) Dermal replacement prepared from mesenchymal cells of hair follicle

(76) Inventors: Jung-Chul Kim, Daegu (KR); Moon-Kyu Kim, Daegu (KR)

Correspondence Address: JONES DAY, 222 EAST 41ST ST, NEW YORK, NY, 10017, US

(21) Appl. No.: 10/499,837

(22) PCT Filed: Dec. 17, 2002
(86) PCT No.: PCT/KR02/02377

(30) Foreign Application Priority Data
Dec. 18, 2001 (KR) 10-2001-0080567

(51) Int. Cl. A61K 45/00; C12N 5/08

(52) U.S. Cl. 424/93.7; 435/371

(57) ABSTRACT

The present invention relates to dermal replacement prepared from mesenchymal cells of hair follicle. As compared with fibroblast, the mesenchymal cells separated from the hair follicle, especially from the hair follicle in human beard produce a lot of growth factor and matrix protein stimulating cell-regeneration, and produce a little enzyme decomposing matrix protein. Therefore, the dermal replacement prepared from the present invention has more excellent cell-regeneration effect than the conventional artificial skin.

### TECHNICAL FIELD

The present invention generally relates to living dermal replacements, and more specifically, to a dermal replacement prepared from mesenchymal cells of hair follicle.

### BACKGROUND ART

Surgical excision of the burn wound and application of an autograft taken from the patient's full-thickness unburned skin is the ideal method to treat patients with deep burn injuries. However, because skin donor sites are first sutured, the amount of self-unburned skin available for wounded skin is very limited. Accordingly, the spilt-thickness dermal graft is regarded as the best treatment method, and either cryopreserved or fresh skin is also used as the current biological dressing for coverage of extensive excised burn wounds (Atnip and Burke, 1983, Curr Prob. Surg. 20:623-83). The performance of cryopreserved allograft skin on wounds is inferior to that of fresh skin, probably due to the loss of viability of keratinocytes and fibroblasts following cryopreservation, freezing and subsequent thawing. In addition, disruption of some of the physical composition of skin, such as basement membrane, by cryopreservation may also contribute to decreased cell viability.

As a result, there have been studied on artificial skins available for the above-described case. Although epidermal and dermal layers of artificial skins are separately developed at first and partially used in clinic trial, problems have been raised. Currently, the method to cover the wounded skin with spilt-thickness graft or with cultured epidermal cells after graft of artificial dermis is used. The artificial dermis is prepared by using sponge or gel type collagen or by using absorbent polymer.

The current technologies on artificial skins are as follows.

1) Cultured Autologous Keratinocyte Graft

The fact that cultured epidermal cells can be used in missing sites of full-thickness skin has been recognized since 1950s. In 1975, Rheinwald and Green reported that epidermal cells were rapidly proliferated when growth-stimulating substances such as epidermal growth factor (EGF) or cholera toxin were added in a culture medium whose bottom was covered with mesenchymal cells. Based on their study, when a small amount of epidermal cells was cultured for 3˜4 weeks, the cells were 5000 times proliferated enough to cover the whole body surface area of adult (1.7 m²).

In order to use the epidermal cells for graft, normal differentiation process should be induced. However, if the epidermal cells are palliatively cultured in a culture medium, they are abnormally differentiated. As a result, they cannot be used for graft. In other words, since stratum corneums are not made, epidermal cells after graft are dried and defected. In addition, since biochemical differentiation incompletely occurs in the cells, the epidermal layers have problems in maintenance of their frames and defense function. Prunieras et al. (1983, J Invest Dermatol 81:28s-33s.) stated that morphological differentiation normally occurred when epidermal layers were exposed to air in their culture step. Maruguchi et al. (1994, Plast Reconstr Surg 93:537-44) reported that biochemical differentiation normally occurred when epidermal cells were cultured on transplanted artificial dermis. Accordingly, they stated that the function and structure of normal epidermis was recovered if epidermal cells were cultured, exposed to air on artificial dermis.

O'connor (1981, Lancet 1:75) first used epidermal cells cultured to burned patients, and grafted the cells in sites of ulcer, nevus, epidermolysis bullosa. O'connor reported that their adhesion rate ranged from 15 to 50%. However, although they adhered well to sites where dermis remained, they did not adhere to fat, chronic wound or infectious wound.

Cultured epidermal cells, even after adhesion, are contracted to 30% of wound size, and hypertrophic scars are more formed than split-thickness graft. In addition, graft sites are easily stripped off or blisters are formed thereon. These phenomena result from unstable epidermal layers and late formation of epidermis-dermis combination sites.

2) Acellular Artificial Dermis

Yannas and Burke (1980, J Biomed Mater Res 14:65-81) developed acellular artificial dermis. The artificial dermis was prepared by mixing glycosaminoglycan in collagen, quickly lyophilizing the mixture and then vacuum-drying it at high temperature. Since wound site had no epidermal layers, a two-step surgical operation should be used. First, wound site was covered with a silastic sheet. Then, when artificial dermis after graft adhered to the wound site, the sheet was removed and the wound site was covered with a spilt-thickness graft.

The artificial dermis has a sponge-type structure having pores. After graft, blood vessel, fibroblast and fibrous tissue are grown into these pores. As a result, a new dermal structure is formed and the artificial dermis becomes combined in normal tissue. Accordingly, the size of pores plays an important role in adhesion of the artificial dermis. The size of pores is dependent on kinds or content of glycosaminoglycan, cross-linkage methods, freezing rate, and concentration of collagen. Suitable size of the pores ranges 50 to 150 μm. The acellular artificial dermis after graft was reported to have a relatively low adhesion rate ranging from 50 to 70%. The low adhesion rate resulted from generation of hematoma in graft sites, high infection rate of 38%, and early degradation by in vivo enzymes. However, the major reason the artificial dermis is not useful is that the frame of the artificial dermis is early degraded by internal collagenase before formation of a structure of new dermis after graft. Although the artificial dermis was used after cross-linked with glutaraldehyde in order to solve this problem, there was another problem in strong cytotoxicity of glutaraldehyde. Another method cells after graft are rapidly proliferated such that new dermis can be quickly formed is to use heparin sulfate having good cytotropism instead of conventional chondroitin-6-sulfate among glycosaminoglycan. Instead of glutaraldehyde, ascorbate-copper ions having no cytotoxicity can be used, but it is difficult to induce a desirable cross-linkage.

3) Cellular Artificial Skin

Cellular artificial dermis is artificial skin having a double-layer structure wherein acellular artificial dermis is covered with cultured epidermal cells in order to solve the problem of the two-step surgery of acellular artificial dermis. Since the artificial dermis has a sponge type wherein cells can penetrate into its pores, the surface of the artificial dermis is covered with collagen gel or sheet, and then epidermal cells are spread thereon. Wound contraction less occurs in this case than in a case wherein acellular artificial dermis is only grafted. It is reported that a structure similar to normal lamina is formed from 11 days after culture.

Cultured fibroblasts are planted in dermis of the artificial skin in order that new dermis after graft can be quickly formed. This method shows an adhesion rate of 70% (Hansbrough, 1989 JAMA 262:2125-30), and 9 days after graft, fixing fibril and basement membrane are formed. Although this method may be used in defective sites of full-thickness skin, it has a problem in toxicity of glutaraldehyde used in cross-linkage of dermal sites.

4) Graft of Cultured Synthetic Skin

Cultured synthetic skin is the artificial skin developed by Bell, known as living skin equivalent or hybrid skin. Epidermis is prepared by culturing epidermal cells on dermal sites of collagen gel type which is prepared by planting and contracting fibroblasts in collagen solution. Fibroblasts of dermal sites increase mechanical tension of artificial skin by maturing collagen gel, make it easy to manipulate, make the artificial skin have a resistance to the collagenase degradation, and stimulate proliferation of epidermal cells. In addition, the fibroblasts generate new stroma, and make cells related to blood vessel and wound healing grow quickly after graft. Accordingly, the fibroblasts have an important role in adhesion of artificial skin. However, there are some problems in Bell's method as follows. The intercellular stroma of dermal sites prepared by Bell's method is irregularly arranged. With the lapse of time, the number of cells decreases on the graft site, and the manipulation during surgery is difficult. After graft, dermal sites are easily degraded, and they have the low adhesion rate of epidermal cells.

5) Artificial Dermis Prepared from Allogeneic Skin

When allogeneic dermis is grafted in defective sites of full-thickness skin and adheres to the skin without rejection, the allogeneic dermis compensates the thickness of dermal layer, thereby obtaining an excellent result as the full-thickness skin is grafted. Immune response of allogeneic skin occurs by cells, and the intercellular stroma of dermis does not cause rejection. Therefore, the allogeneic dermis can be used for graft when cells are all removed and freeze-dried to maintain the structure of intercellular stroma. The allogeneic dermis processed by the above-described method has been marketed as a product, AlloDerm. However, the product is expensive, and is dependent on production system by order which patients are rapidly provided with living cell tissues mass-cultured in an aseptic room according to doctor's prescription. Accordingly, products developed in foreign countries can have a problem in cell necrosis phenomenon due to a long-period process of providing patients with them.

6) Artificial Dermis Prepared from Biodegradable Polymer

After Langer and Vacanti introduce a tissue engineering technology for generating desired tissues by using absorbent polymer, the technology is applied to artificial skin. The artificial dermis prepared from biodegradable polymer is the dermis prepared by planting fibroblasts in framework formed with polymer instead of collagen in order to solve problems of artificial dermis made of collagen. Inflammation is generated on the artificial skin made of collagen after graft, and the artificial skin is dissolved before formation of new frame of dermis. Artificial dermis prepared by using polyglactin in American Advanced Tissue Science is marketed as Dermagraft and granted a patent as the U.S. Pat. No. 5,460,939. Since Dermagraft is covered with a silastic sheet, vascularization is completed 2 weeks after graft of Dermagraft in living body. Accordingly, if the silastic sheet is removed and the removed site is covered with split-thickness skin, its adhesion rate is 51%. Although polyglactin is degraded in living body by hydrolysis within 60 days, it begins being degraded during a culture period in a culture medium when artificial dermis is prepared. As a result, since polyglactin after graft is quickly dissolved and removed, it does not serve as a function of artificial skin well.

### DETAILED DESCRIPTION OF THE INVENTION

The present invention has an object to provide a dermal replacement including a living stromal tissue cultured in a three-dimensional framework and a transitional covering.

The disclosed stromal tissue comprises mesenchymal cells of hair follicle, extracellular matrix proteins and growth factors secreted from the mesenchymal cells.

The mesenchymal cells of hair follicle are dermal papilla cells and connective tissue sheath cells.

The dermal papilla 100 has long been regarded as a prerequisite for hair growth initiation and maintenance. However, the function of the connective tissue sheath 102 which surrounds the lower segment of a follicle and contains a vascular plexus, is unknown (see FIG. 1).

Although all mesenchymal cells of hair follicle can be used for the disclosed mesenchymal cells of hair follicle, mesenchymal cells of scalp or beard follicle are preferable. Mesenchymal cells of beard follicle are used in the Examples of the present invention.

Referring to reference example 1, α-smooth muscle protein SM22 and α-smooth muscle actin distinctly existing in myofibroblasts are detected in mesenchymal cells of hair follicle but not in fibroblasts. Accordingly, the disclosed mesenchymal cells of hair follicle have characteristics closer to myofibroblasts than to fibroblasts.

The most important things of dermal replacement are matrix proteins such as collagen, fibronectin, decorin and osteonectin, and growth factors such as connective tissue growth factor, pigment epithelium derived factor, platelet derived growth factor, insulin-like growth factor, transforming growth factor and glycosaminoglycan. Referring to reference example 2, the production of matrix proteins and growth factors in mesenchymal cells of hair follicle is higher than fibroblasts used for stromal cells of prior art. However, collagenase activity in the mesenchymal cells of hair follicle which degrades the matrix proteins, is less than fibroblasts. Thus, the present invention can provide a dermal replacement having an excellent ability of regenerating skin cells by using the mesenchymal cells of hair follicle.

The present invention includes a three-dimensional living stromal tissue connected to a transitional covering as a framework.

The transitional covering is formed of silicone rubber such as polyurethane and silastic sheet.

The three-dimensional framework allows cells to attach to it and grow in more than one layer. A non-biodegradable material such as nylon (polyamide), dacron (polyester), polystyrene, polypropylene, polyacrylate, polyvinylchloride (PVC), polycarbonate (PC) and nitrocellulose may be used to form the framework. For in vivo use, it is preferable to use a biodegradable framework such as polyglactic acid, polyglucuronic acid, collagen, fibrin, gelatin, cotton, cellulose, chitosan or dextran.

In the examples of the present invention, a dermal replacement is prepared by culturing mesenchymal cells separated from beard in a three-dimensional framework formed of collagen-chitosan-glycosaminoglycan.

### BRIEF DESCRIPTION OF THE DRAWINGS

FIG. 1 shows a structure of hair follicle.

FIG. 2a is a picture showing patterns of mesenchymal cells of hair follicle in their culture state.

FIG. 2b is a picture showing patterns of fibroblasts in their culture state.

FIG. 3 is a graph illustrating growth rate when the mesenchymal cells of hair follicle and fibroblasts are cultured for 10 days.

FIG. 4a is a picture showing a result of immunostaining mesenchymal cells of hair follicle in their culture state by using anti-α-smooth muscle actin antibody.

FIG. 4b is a picture showing a result of immunostaining fibroblasts in their culture state by using anti-α-smooth muscle actin antibody.

FIG. 5 is a picture showing a result of culturing mesenchymal cells of beard in a three-dimensional framework.

FIG. 6 is a picture showing a result of staining the mesenchymal cells of beard cultured in the three-dimensional framework.

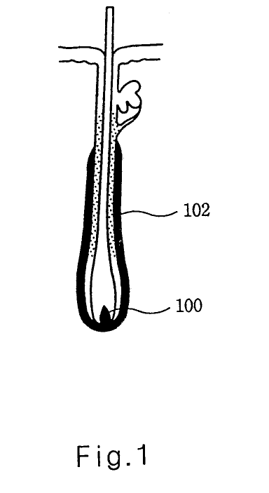

### PREFERRED EMBODIMENTS OF THE INVENTION

### REFERENCE EXAMPLE 1

### Experiment on Taxonomic Difference Between Mesenchymal Cells of Hair Follicle and Fibroblasts

1) Culture of Mesenchymal cells of Hair Follicle and Fibroblasts

Beard tissues were obtained from a male alopecia patient by biopsy to separate hair follicle of beard. ⅔ of the upper portion of the separated hair follicle was removed, and the rest ⅓ of the lower portion was cultured in 5% carbon dioxide. Fibroblasts were obtained from the skin in circumcision. Dulbecco's modified Eagle's Medium (DMEM; Gibco BRL, Gaithersburg, Md., USA) including penicillin (100 U/ml), streptomycin (100 ug/ml), glutamine (0.584 mg/ml), and 20% Fetal Bovine Serum is used as liquid medium. The liquid medium was changed every three days. 4 weeks after culture, each cell was isolated with 0.25% trypsin and 0.02% EDTA solution, and then sub-cultured. The cell growth rate for 10 days was measured by using the third sub-cultured cell.

2) Immunohistochemistry

Cell cultures at passage 3 were fixed for 5 minutes in methanol and incubated with a monoclonal antibody to α-smooth muscle actin at room temperature for 1 hour. After thorough washing in PBS (phosphate-buffered saline), cells were exposed to biotinylated anti-mouse antibody (Dako, Glostrup, Denmark) for 1 hour, washed again, and incubated with horseradish peroxidase-linked steptavidin for the same length of time. Color was developed with 1% hydrogen peroxide and 5% diaminobenzidine. After immunostaining, some sections were lightly counterstained with hematoxylin before dehydration and mounting.

The results of the above-described experiments are as follows.

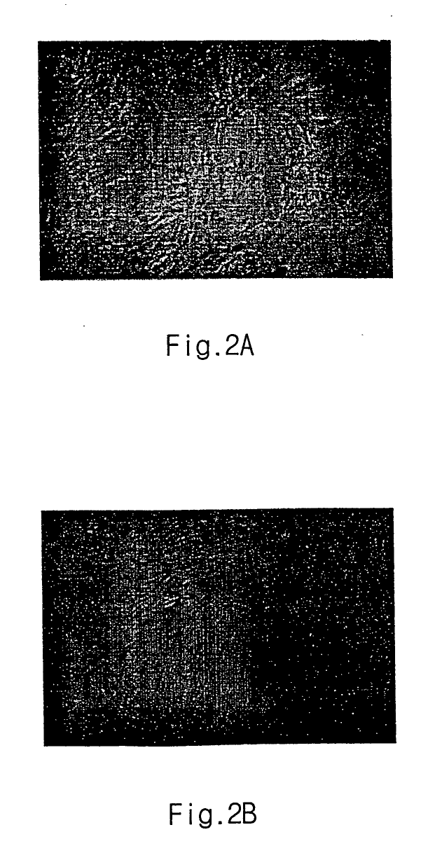

As shown in FIGS. 2a and 2b, mesenchymal cells of beard assumed a flattened morphology with numerous cell processes whereas nonfollicular dermal fibroblasts had a more regular, spindle-shaped morphology. The mesenchymal cells formed clumps or aggregates. This aggregation contrasted with the regular patchwork patterning of skin fibroblasts.

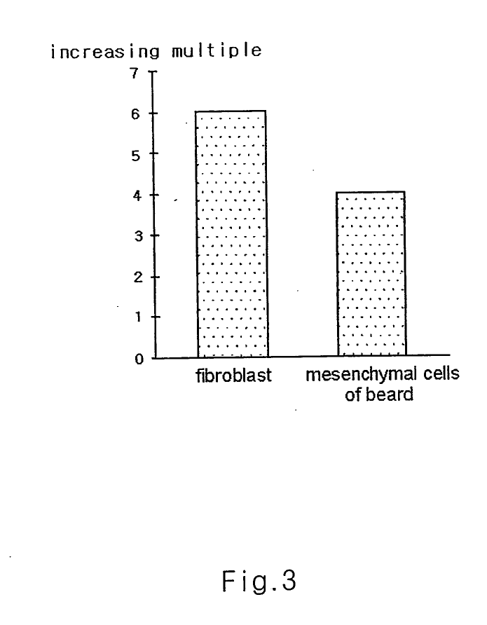

As shown in FIG. 3, mesenchymal cells of beard grew slower than dermal fibroblasts.

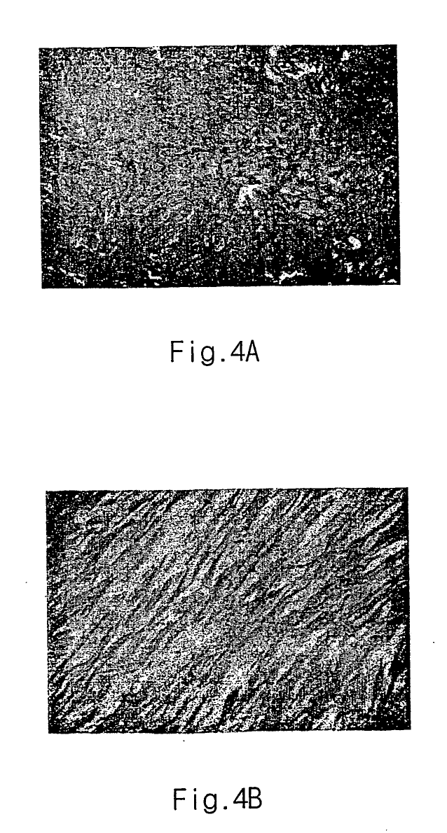

Referring to FIGS. 4a and 4b, when fibroblasts and mesenchymal cells of beard were immuno-stained with anti-α-smooth muscle actin antibody, the fibroblasts were rarely stained but mesenchymal cells of hair follicle were clearly stained. This result suggested that mesenchymal cells of beard were closer to myofibroblasts than to fibroblasts.

### REFERENCE EXAMPLE 2

### Construction of cDNA Library from Mesenchymal Cells of Hair Follicle and Fibroblasts, and Experiment on Frequency Difference in Gene Expression Through cDNA Analysis

Mesenchymal cells of hair follicle and dermal fibroblasts were cultured in DMEM, and poly(A)+RNA was prepared from 70% confluent cells. A cDNA library was constructed in the ZAP II vector (Stratagene, USA) by use of poly(A)+RNA (5 μg) and Uni-Zap XR kit (Stratagene). The phage library was converted into a pBluescript phagemid cDNA library by in vivo excision by the ExAssist/SOLR system (Stratagene). The pBluescript cDNA library was plated on LB plates with X-gal, IPTG, and ampicillin, and white colonies were selected for sequencing.

Overnight cultures (3 ml in LB) of selected clones were used to prepare plasmid DNA by QIAwell-8 plasmid mini-extraction kits (QIAGEN, Chatsworth, Calif.). cDNAs were sequenced from 5′ end of the inserts using a Sequenase DNA sequencing kit. Sequences were compared with GenBank data base.

1400 clones from each cDNA library were analyzed to compare genes of matrix proteins, growth factors and enzymes degrading matrix protein. The results were shown in Table 1.

TABLE 1
Frequency of gene expression of growth factor, matrix protein
and enzyme degrading matrix protein in mesenchymal cells separated from
hair follicle and fibroblasts separated from the skin

| | | Mesenchymal | |
|---|---|---|---|
| | | cells of hair | |
| | | follicle | fibroblasts |
| Growth factor | Connective tissue | 29 | 0 |
| Gene | growth factor | | |
| | Pigment epithelial- | 4 | 0 |
| | differentiation factor | | |
| | Cyr61 | 5 | 0 |
| | IGF-2 | 2 | 0 |
| | Mac-25 | 6 | 0 |
| Extracellular matrix | Fibronectin | 39 | 20 |
| gene | Type I collagen | 34 | 4 |
| | Osteonectin | 31 | 6 |
| | Decorin | 9 | 0 |
| Enzyme | Stromelysin | 0 | 22 |
| decomposing | collagenase | 0 | 13 |
| extracellular matrix | | | |

### EXAMPLE

### Preparation of Dermal Replacement Using Mesenchymal Cells of Hair Follicle

1) Culture of Mesenchymal Cells from Beard

Beard tissues were obtained from a male alopecia patient to separate hair follicle of beard. ⅔ of the upper portion of the separated hair follicle was removed, and the rest ⅓ of the lower portion was cultured in 5% carbon dioxide at 37° C. Dulbecco's modified Eagle's medium (DMEM; Gibco BRL, Gaithersburg, Md., USA) containing penicillin (100 U/ml), streptomycin (100 ug/ml), glutamin (0.584 mg/ml) and 20% Fetal Bovine Serum is used as culture solution. The medium was changed every 3rd day. 4 weeks after culture, each cell was isolated with 0.25% trypsin and 0.02% EDTA solution, and then sub-cultured.

2) Preparation of Dermal Replacement

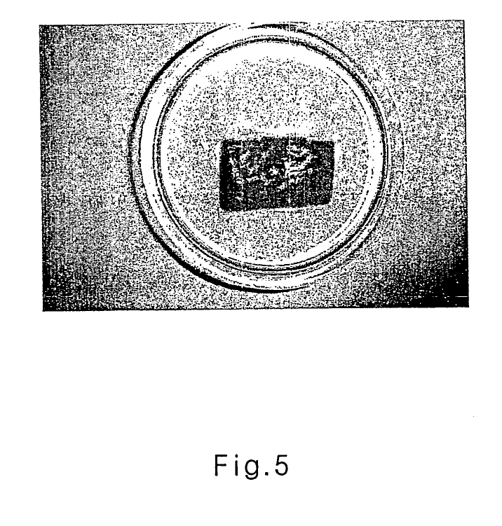

Referring to FIG. 5, a collagen-chitosan-glycosaminoglycan sheet was cut by 5×8 cm. 5×10⁵ of the cultured mesenchymal cells of hair follicle were placed on the sheet, and cultured for 4˜5 weeks.

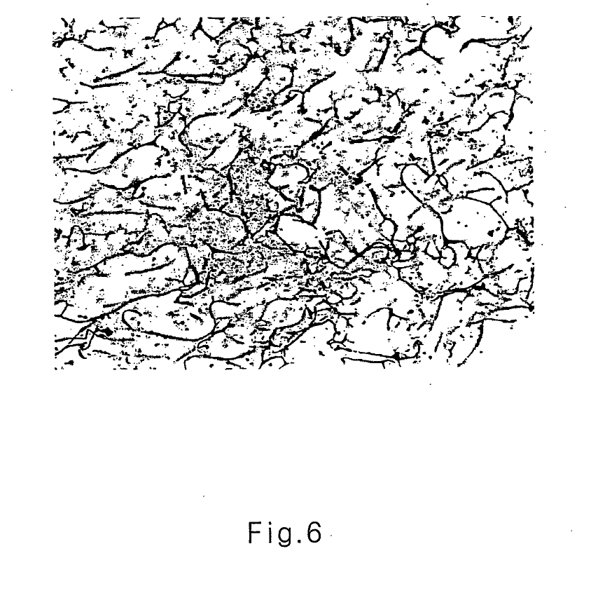

Referring to FIG. 6, it was shown that the mesenchymal cells of hair follicle attached to the three-dimensional framework and they were well cultured.

### Industrial Applicability

As discussed earlier, mesenchymal cells of hair follicle produce more growth factors and matrix proteins which stimulate cell-regeneration than fibroblasts while producing less enzymes which degrade matrix proteins than fibroblasts. Accordingly, the disclosed dermal replacement prepared using mesenchymal cells has more excellent effect of cell-regeneration than the conventional art.

What is claimed is:

1. A dermal replacement comprising:
a) a living stromal tissue including mesenchymal cells of hair follicle cultured in a three-dimensional framework and connective tissue proteins secreted from the mesenchymal cells of hair follicle; and
b) a transitional covering connected to the stromal tissue.

2. The dermal replacement according to claim 1, wherein the mesenchymal cells of hair follicle are dermal papilla cells and connective tissue sheath cells.

3. The dermal replacement according to claim 1 or 2, wherein the hair follicle is scalp or beard follicle.

4. The dermal replacement according to claim 1, wherein the three-dimensional framework is composed of one or more biodegradable material selected from the group consisting of polyglactic acid, chitin, chitosan, cotton, polyglucuronic acid, cellulose, gelatin, collagen, fibrin and dextran.

5. The dermal replacement according to claim 1, wherein the framework is composed of one or more non-biodegradable material selected from the group consisting of polyamide, polyester, polystyrene, polypropylene, polyacrylate, polyvinylchloride, polycarbonate, polytetrafluoroethylene and nitrocellulose.

6. The dermal replacement according to claim 1, wherein the transitional covering is made of silicone or polyurethane.
